(19) United States
(12) Patent Application Publication
(10) Pub. No.: US 2026/0098009 A1
(43) Pub. Date: Apr. 9, 2026

(54) Process for the Production of Specialty Mercaptans From the C4 Oligomerization Product

(71) Applicant: Indian Oil Corporation Limited, Mumbai (IN)

(72) Inventors: Vimal Kumar Upadhyay, Faridabad (IN); Saravanan Subramani, Faridabad (IN); Satyen Kumar Das, Faridabad (IN); Seema Yadav, Faridabad (IN); Sarvesh Kumar, Faridabad (IN); Madhusudan Sau, Faridabad (IN)

(73) Assignee: IndianOil Corporation Limited, Mumbai, MH (IN)

(21) Appl. No.: 19/353,599

(22) Filed: Oct. 8, 2025

(30) Foreign Application Priority Data
Oct. 9, 2024 (IN) 202421076612

(51) Int. Cl. C07C 319/04

(52) U.S. Cl. CPC C07C 319/04

(57) ABSTRACT

The present invention relates to a process for the production of specialty mercaptans from C₄ oligomerization product. The oligomer product from the commercial C₄ oligomerization process is separated in desired fractions of C₈ and C₁₂ olefins and these streams are further used as feed stock for the production of the specialty mercaptans such as tert octyl mercaptan (TOM) & tert dodecyl mercaptan (TDM). These fractionated oligomer stream (C₈ and C₁₂) from the C₄ oligomerization process reacts with hydrogen sulfide in presence of pretreated cation exchange resin catalyst using appropriate inhibitors such as amine precursors, nitriles and metals to produce respective sulfur compounds tert-octyl mercaptan (TOM) and tert dodecyl mercaptan (TDM).

This application claims priority to Indian Application No. 202421076612 dated Oct. 9, 2024. The contents of this application are hereby incorporated by reference in its entirety.

### FIELD OF THE INVENTION

The present invention relates to a process for the production of specialty mercaptans from the C₄ oligomerization product. Particularly, C₄ oligomerization product separated in desired fractions are reacted with hydrogen sulfide in presence of pretreated cation exchange resin catalyst to produce respective specialty sulfur compounds/mercaptan such as tert-octyl mercaptan (TOM) & tert dodecyl mercaptan (TDM).

### BACKGROUND OF THE INVENTION

The C₄ oligomerization process is a well-established method for producing high-purity di-isobutylene (DIB), a C₈ olefin widely recognized for its value as a gasoline additive due to its high octane rating. However, during this process, a certain amount of tri-isobutylene (TIB), a C₁₂ olefin, is also formed as an undesired byproduct. Currently, C₁₂ oligomers are incorporated into gasoline blends, but their presence can elevate the final boiling point (FBP) of the fuel, limiting the quantities that can be blended without negatively impacting gasoline quality.

Despite the advantage and limitation of DIB and TIB respectively from the C₄ oligomerization process, these products have significant potential to be valorised into valuable specialty chemicals, specifically tert-octyl mercaptan (TOM) and tert-dodecyl mercaptan (TDM). Converting DIB into value-added chemicals like TOM can diversify its applications beyond gasoline, offering flexibility in the C₄ oligomerization process and enhancing its economic value. Similarly, TIB which is an undesired byproduct, can be effectively utilized in the production of specialty product TDM.

Currently elemental sulfur is recovered from the H₂S gas via clause process, which is a highly energy intensive process. The recovered elemental sulfur is a low value product of the refinery. The synthesis of mercaptans using catalytic methods has been explored in prior art, some of which are referenced below.

U.S. Pat. No. 4,102,931A describes synthesis process of various mercaptans using zeolite based catalyst and oligomer feed streams ranging from C₄ to C₁₂ reacted with hydrogen sulfide to produce respective tertiary mercaptans in presence of synthetic zeolite based catalyst. The operating conditions used are temperature 50-150° C., pressure 20-1000 psig and oligomer to H₂S mole ratio of 20-250. The process finds performance of synthetic zeolite catalyst is superior to other conventional homogeneous catalyst and effect of moisture in the feed. Presence of small amount of moisture degrades the catalyst performance.

U.S. Pat. No. 4,565,893A discloses the production of tert-dodecyl mercaptan using tetra-propene or tri-isobutene as a feed stock and cation exchange resin catalyst. Conditions applied for synthesis 45-75° C. temperature, 1-50 bar pressure and 1.2-10 mole ratio of H₂S to tetra-propene in a continuous tubular reactor. Effect of moisture in the yield and selectivity of the TDM is also studied in this invention. The process finds out that presence of the moisture in the catalyst will reduce the catalyst activity after 100 hours of operation and also discloses that cation exchange resin employed for TDM synthesis should be sufficiently dry before use and moisture should be <0.5 wt %. The catalyst should be dried at 80° C. for at least 8 hours prior to use.

U.S. Pat. No. 4,582,939A discloses synthesis of tertiary mercaptans using sulfonic acid based cation exchange resin catalyst. The olefinic feed stream typically consists of isobutylene is reacted with hydrogen sulfide (H₂S) at 60-110° C. The H₂S to olefins mole ration used 1:1 and pressure 1-5 atmosphere. This process finds out that catalyst activity degrades due to deposition of the higher oligomer on the catalyst surface. The higher oligomer is a byproduct of the main reaction. Further this process describes that catalyst activity may be rejuvenate by backwashing with aliphatic hydrocarbons such as heptane at 90° C. for 2-4 hours.

U.S. Pat. No. 6,544,936B2 discloses process for production of tertiary mercaptans using C₄ oligomer feed stocks such as isobutylene, di-isobutylene, tri-isobutylene, tri-propylene, tetra-propylene etc using small pore size synthetic zeolite catalyst. In this process Y type Zeolite catalyst with a medium or large pore size in the range of 0.5-0.8 nm is employed and synthesis is carried out at 70-150° C. temperature, 1-20 bar pressure and H₂S to olefin mole ratio of 0.5-5.

US2010/0249366A1 discloses synthesizes of tert dodecyl mercaptan from tri-butene using cation exchange resin. This process further discloses the application of TDM as chain modifier in radial polymerization process to produce polysulfides. The TDM is produced at 70-120° C., 10-20 bar pressure and H₂S to olefin mole ratio of 1-5 in continuous mode operation.

U.S. Pat. No. 3,661,745A discloses synthesis of TDM by using high energy ionization radiation in a tubular reactor. Equimolar mixture of dodecene and hydrogen sulfide is used for the synthesis of TDM. In this process reaction temperature maintained in the range 0-50° C. and the reaction pressure about 20 atmospheres.

US20100249366A1 discloses a radical polymerization process in which tert-dodecyl mercaptan is prepared by reaction of hydrogen sulfide with tri (n-butene) in the presence of a cation exchange catalyst, at 19 millibar, and 123° C.±1° C.

In conventional processes various types of catalysts such as zeolite, cation exchange resin, homogeneous catalysts and heterogeneous catalysts are directly used without any pretreatment for the synthesis of TOM/TDM. As a result, due to high activity of the catalyst, heavier oligomer formation takes place as a byproduct, which deposits on the catalyst surface and reduces the catalyst activity. The heavier oligomers also create additional load in the product purification process. So, there is a need to develop a process by optimizing the activity of the catalyst to increase the selectivity of the targeted product and reduce the byproducts. The present invention addresses the problems of the prior art by providing a process for production of highly selective TOM/TDM using pretreated cation exchange resin catalyst to optimize the active sites of the catalysts using appropriate inhibitors such as amine precursors, nitriles, various metals for better yield and selectivity of TOM/TDM. The optimized activity of the catalyst contributes very less oligomer formation which minimizes the deactivation rate of the catalyst.

### OBJECTIVE OF THE INVENTION

It is a primary objective of the present invention to provide a process for production of tert octyl mercaptan & tert dodecyl mercaptan.

It is another objective to provide a process for production of high purity tert octyl mercaptan & tert dodecyl mercaptan using pretreated cation exchange resin catalyst.

It is another objective to enhance the catalyst activity towards selective production of specialty mercaptans by optimizing active sites of the catalysts using appropriate inhibitors such as amine precursors, nitriles and various metals.

It is another objective of the present invention to provide a process for improving yield/selectivity of tert octyl mercaptan & tert dodecyl mercaptan.

Another objective of the present invention is to reduce the formation of undesired oligomer by-products.

Another objective of the present invention is to enhance the performance of the cation exchange resin catalyst and also extend its life by optimizing catalyst activity which reduces its deactivation.

A yet another objective of the present invention is to provide a process for production of high purity tert octyl mercaptan & tert dodecyl mercaptan that is more efficient and environmentally friendly.

### SUMMARY OF THE INVENTION

In an embodiment, the present invention provides a process for production of mercaptans from C₄ oligomerization product, the process comprising: fractionating C₄ oligomerization product to obtain oligomer products di-isobutene (DIB) and tri-isobutene (TIB); loading a cation exchange resin catalyst into a reactor; pretreating the loaded cation exchange resin catalyst with an inhibitor in the reactor; feeding the oligomer products di-isobutene or tri-isobutene and hydrogen sulfide (H₂S) into the reactor loaded with the pretreated cation exchange resin catalyst to obtain a reaction product mixture; and separating the reaction product mixture to obtain un-converted oligomer and tert octyl mercaptan or tert dodecyl mercaptan.

In an embodiment of the present invention, there is provided a process, wherein the cation exchange resin catalyst is pretreated with 2-10 wt % of the inhibitor, followed by heating at a temperature in a range of 50 to 90° C. for 3 to 4 hours and drying of catalyst at a temperature in a range of 100 to 120° C. for 5 to 10 hours in the presence of nitrogen (N₂).

In an embodiment of the present invention, there is provided a process, wherein oligomer products di-isobutene or tri-isobutene and hydrogen sulfide (H₂S) are mixed and reacted into the reactor loaded with the pretreated catalyst at a temperature in a range of 50 to 110° C., pressure of 1 to 15 bar and H₂S to di-isobutylene/tri-isobutylene mole ratio of 1:1 to 10:1.

In an embodiment of the present invention, there is provided a process, wherein the inhibitor is selected from an amine precursor, nitrile precursor and metal precursor, wherein the amine precursor is methyl ethanol amine or methyl di ethanol amine, the nitrile precursor is acetonitrile and metal precursor are iron or sodium.

In an embodiment of the present invention, there is provided a process, wherein the cation exchange resin catalyst is selected from strongly acidic cation exchange resins, wherein the acidic cation exchange resin is sulfonated polystyrene divinyl benzene resin.

In an embodiment of the present invention, there is provided a process, wherein the pretreated cation exchange catalyst has an active site concentration in a range of 3 to 4.5 eq/kg.

In an embodiment of the present invention, there is provided a process, wherein the pretreated cation exchange catalyst has selectivity for tert octyl mercaptan or tert dodecyl mercaptan in a range of 75% to 95%.

In an embodiment of the present invention, there is provided a process, wherein the di-isobutene conversion to tert octyl mercaptan is in a range of 45% to 98%.

In an embodiment of the present invention, there is provided a process, wherein the tri-isobutene conversion to tert dodecyl mercaptan is in a range of 5% to 10%.

In an embodiment of the present invention, there is provided a process, wherein the tert octyl mercaptan has purity in a range of 75% to 99%, wherein the tert dodecyl mercaptan has purity in a range of 90% to 95%.

### BRIEF DESCRIPTION OF THE ACCOMPANYING DRAWINGS

The following figures form part of the present specification and are included to further illustrate aspects of the present disclosure. The disclosure may be better understood by reference to the figures in combination with the detailed description of the specific embodiments presented herein.

FIG. 1 illustrates schematic representation of process to produce TOM/TDM, according to an embodiment of the present disclosure.

FIG. 2 illustrates schematic representation of catalyst activity for longer life in terms of TOM yield.

### DETAILED DESCRIPTION OF THE INVENTION

For convenience, before further description of the present disclosure, certain terms employed in the specification, and examples are delineated here. These definitions should be read in light of the remainder of the disclosure and understood as by a person of skill in the art.

The terms used herein have the meanings recognized and known to those of skill in the art, however, for convenience and completeness, particular terms and their meanings are set forth below. The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”. The term “at least one” is used to mean one or more and thus includes individual components as well as mixtures/combinations. Throughout this specification, unless the context requires otherwise the word “comprise”, and variations such as “comprises” and “comprising”, will be understood to imply the inclusion of a stated element or step or group of elements or steps but not the exclusion of any other element or step or group of element or steps. The term “including” is used to mean “including but not limited to”. “including” and “including but not limited to” are used interchangeably.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods and materials are now described.

The present disclosure is not to be limited in scope by the specific embodiments described herein, which are intended for the purposes of exemplification only. Functionally equivalent products, compositions, and methods are clearly within the scope of the disclosure, as described herein.

The present invention provides the process to produce high purity tert octyl mercaptan & tert dodecyl mercaptan using cation exchange resin catalyst. The invention also provides a process scheme for valorization of C₄ streams to value added products such as tert dodecyl mercaptan & tert octyl mercaptans. These can be used in styrene butadiene polymerization process as chain modifiers and it can be used in synthetic lubricants for the production of high wear & tear lubricants.

In the present invention the cation exchange resin catalyst activity is optimized by pretreatment of the catalyst using inhibitors such as amine precursor, nitrile and metals etc. The pretreatment of catalyst reduces the formation of undesired heavier oligomers and enhances the yield and selectivity of the product. The undesired heavier oligomer generally deposits on the catalyst surface and deteriorates the catalyst performance. The technical advantage of pretreatment of catalyst are as follows:
The formation of heavier oligomers was suppressed by 10-30% for TOM/TDM synthesis
Less undesired products increase product selectivity and purity. Additionally, it will reduce the load on the product purification.

No catalyst activity deterioration takes place due to less formation of undesired by-products.

In an aspect, the present invention provides a process for production of mercaptans from C₄ oligomerization product, the process comprising:
a. fractionating C₄ oligomerization product to obtain oligomer products di-isobutene (DIB) and tri-isobutene (TIB);
b. loading a cation exchange resin catalyst into a reactor;
c. pretreating the loaded cation exchange resin catalyst with an inhibitor in the reactor;
d. feeding the oligomer products di-isobutene or tri-isobutene and hydrogen sulfide (H₂S) into the reactor loaded with the pretreated cation exchange resin catalyst to obtain a reaction product mixture; and
e. separating the reaction product mixture to obtain un-converted oligomer and tert octyl mercaptan or tert dodecyl mercaptan.

In an embodiment of the present invention, there is provided a process, wherein the cation exchange resin catalyst is pretreated with 2 to 10 wt % of the inhibitor, followed by heating at a temperature in a range of 50 to 90° C. for 3 to 4 hours and drying of catalyst at a temperature in a range of 100 to 120° C. for at least 5 to 10 hours in the presence of nitrogen (N2).

In an embodiment of the present invention, there is provided a process, wherein oligomer products di-isobutene or tri-isobutene and hydrogen sulfide (H₂S) are mixed and reacted into the reactor loaded with the pretreated catalyst at a temperature in a range of 50 to 110° C., pressure of 1 to 15 bar and H2S to di-isobutylene/tri-isobutylene mole ratio of 1:1 to 10:1.

In an embodiment of the present invention, there is provided a process, wherein the cation exchange resin catalyst is selected from strongly acidic cation exchange resins, wherein the acidic cation exchange resin is sulfonated polystyrene divinyl benzene resin.

In an embodiment of the present invention, there is provided a process, wherein the inhibitor is selected from amine precursor, nitrile precursor and metal precursor, particularly amine precursor is methyl ethanol amine or methyl di-ethanol amine, more particularly methyl di-ethanol amine (MDEA).

In an embodiment of the present invention, there is provided a process, wherein the nitrile precursor is acetonitrile and metal precursor are iron or sodium.

In an embodiment of the present invention, there is provided a process, wherein the pretreated cation exchange catalyst has an active site concentration in a range of 3 to 4.5 eq/kg.

In an embodiment of the present invention, there is provided a process, wherein the pretreated cation exchange catalyst has selectivity for tert octyl mercaptan or tert dodecyl mercaptan in a range of 75% to 95%.

In an embodiment of the present invention, there is provided a process, wherein the di-isobutene conversion to tert octyl mercaptan is in a range of 45% to 98%.

In an embodiment of the present invention, there is provided a process, wherein the tri-isobutene conversion to tert dodecyl mercaptan is in a range of 5% to 10%.

In an embodiment of the present invention, there is provided a process, wherein the tert octyl mercaptan has purity in a range of 75% to 99%, wherein the tert dodecyl mercaptan has purity in a range of 90% to 95%.

The production of TOM and TDM involves reacting their respective olefinic streams (C₈ for TOM and C₁₂ for TDM) with hydrogen sulfide (H₂S) gas. H₂S, a low-value byproduct of the petroleum refining industry, is typically produced from various desulfurization processes, such as hydrocracking, hydrotreating, and amine absorption.

The proposed method not only provides a sustainable use for low-value hydrogen sulfide but also effectively valorizes the heavier, undesired oligomers from the C₄ oligomerization process. This dual approach offers significant value addition and profitability while broadening the scope of the process to produce specialty chemicals.

C₄ oligomerization is well known process for the production of di-isobutylene which is a high octane gasoline additive. In the oligomerization process formation of higher oligomers such as tri-isobutene (10-15 wt %) also takes place which is highly undesired product. The oligomer product reacts with hydrogen sulfide in the presence of cation exchange resin catalyst. Synthesis/process conditions are given in Table-1. During the synthesis of these specialty mercaptans, formation of higher oligomer also takes place which gets deposited on the catalyst surface and reduces the catalyst activity.

TABLE 1
Operating Conditions

| S. No | Parameter |
|---|---|
| Temp(° C.) | 50-110 |
| Pressure (bar) | 1-15 |
| H₂S/Oligomer Product Mole Ratio | 10-1 |

In the present invention the catalyst is pretreated prior to use. The pretreatment will reduce the catalyst activity and reduce the heavy oligomers formation during the TOM/TDM synthesis, which increases the product selectivity and also reduces the catalyst deactivation rate. The pretreatment conditions are given in Table 2. High selectivity and purity of product streams reduces the additional load on separation process in purification section.

TABLE 2
Catalyst Pretreatment

| S. No | Parameter | |
|---|---|---|
| Inhibitor concentration | 2-10 | wt % |
| Pre-treatment Temperature | 70-90° | C. |
| Pretreatment Time | At least 3 hours, preferably 4-8 hours | |
| Drying temperature | 100-120° | C. |
| post pretreatment | | |

| | |
|---|---|
| Drying Time | At least 8 hours in presence of N₂ |

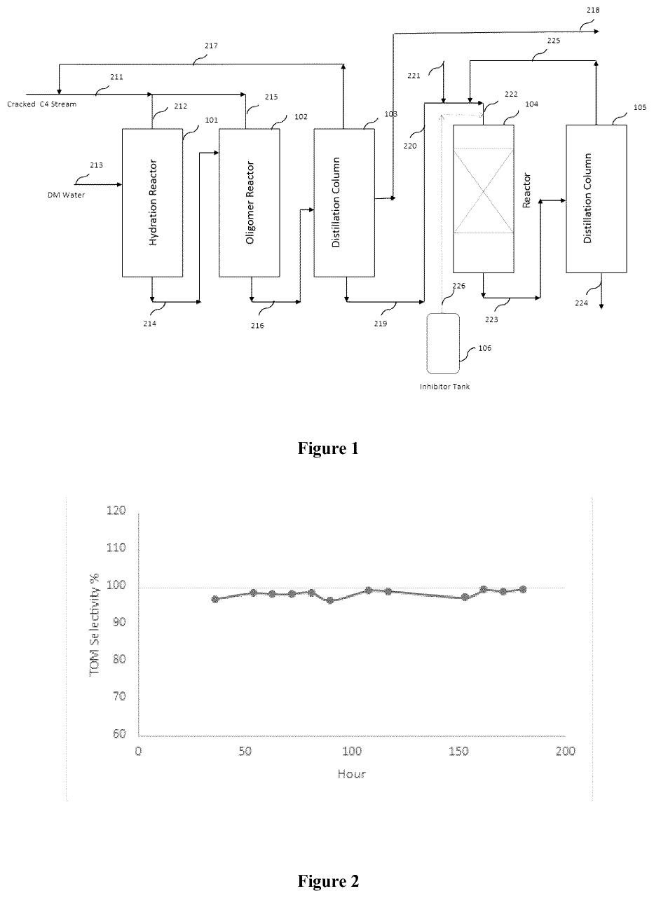

The schematic representation of the present invention for the production of specialty mercaptans from the C₄ oligomerization process is given in FIG. 1. FIG. 1 shows commercial C₄ oligomerization process which consists of a hydration reactor (101) along with the oligomer reactor (102). The C₄ stream (210) and unconverted recycle C₄ stream (217) is mixed up and partially sent to hydration reactor (101) & oligomerization reactor where tert-butyl alcohol (TBA) & C₄ oligomerization takes place respectively. The product from the hydration reactors which consists of tert-butanol is sent to the oligomerization reactor (102) via stream (214). TBA acts as additive to restrict formation of higher oligomers of C₄ mainly trimers and tetramers formation. Further, the product stream (216) from the oligomer reactor is fractionated in fraction column (103) where unconverted C₄ (217) is separated from the top recycled back. The di-isobutene (DIB) rich stream (218) is separated in the middle stream and it is rooted to the gasoline pool. Tri-isobutene (TIB) rich oligomer product stream (219) is separated from the bottom which is further taken in the reactor for synthesis of specialty mercaptans. These DIB & TIB rich streams are further used as feed for the production of tert octyl mercaptan (TOM) or tert dodecyl mercaptan (TDM) based on the requirement in a separate reactor (104) where pretreated catalyst is used for the reaction. Before proceeding for TOM/TDM synthesis the catalyst is pretreated with appropriate inhibitors such as amine precursors, nitriles and various metals from the inhibitor storage tank (106) via stream (226). In this process catalyst bed is soaked in inhibitor solution for at least 3 hours as per the pretreatment conditions. After completion of the catalyst soaking, inhibitor solution is drained, and catalyst is dried as per disclosed conditions. Further pretreated dried catalyst is used for the synthesis of the TDM/TOM as per disclosed process conditions. The oligomer products from fractionators (103) and hydrogen sulfide (H₂S) via stream (221) is taken to the reactor (104). Product stream (223) from reactor is routed to commercial fractionator (105) at vacuum conditions where un-converted oligomers (225) are separated and recycled back to the reactor to optimize the conversion. The bottom stream (224) from fractionator column (105) contains high purity TOM/TDM which is further routed to a storage tank.

### EXAMPLES

The present disclosure is further illustrated by reference to the following examples which is for illustrative purposes only and does not limit the scope of the disclosure in any way. These examples are not intended to be inclusive of all aspects of the subject matter disclosed herein, but rather to illustrate representative features, methods, compositions, and results. These examples are not intended to exclude equivalents and variations of the present disclosure, which are apparent to one skilled in the art.

### Example 1

In this example, the cation exchange resin catalyst is pretreated with different inhibitors and the pretreated catalyst performance has been evaluated for the synthesis of tert-octyl mercaptan. Pre-treatment has been carried out at 70-90° C., 2% by weight of inhibitor solutions used for the catalyst pretreatment. After the pretreatment of the catalyst, synthesis of tert-octyl mercaptan was carried out in the fixed bed process at 60-80° C. and 5-15 bar pressure conditions. Di-isobutylene (DIB) with a purity of 98% and hydrogen sulfide (H₂S) with a purity of 99.9% were employed as the feedstocks. H₂S and DIB were introduced in the reactor after proper mixing, ensuring a mole ratio of H₂S to DIB of 2:1. The outcome of the experiments has been given in Table-3. With using the different precursor for the catalyst treatment, it is observed that only treatment with the methyl di-ethanol amine shows the catalyst activity, no conversion is observed using other precursors, this may be attributed either deactivation of the catalyst activity or the clogging of the pore size of the catalyst.

TABLE 3
Effect of catalyst pretreatment using
different precursors on TOM synthesis

| | Inhibitor | Pretreated | | % Tert |
|---|---|---|---|---|
| | Precursor used | Catalyst | % Conversion | Octyl |
| S. | for catalyst | Activity (Acid | of di- | mercaptan |
| NO | pretreatment | Capacity), meq/g | isobutylene | selectivity |
| 1. | Methyl Ethanol | 3.31 | No conversion | — |
| | Amine | | | |
| 2. | Methyl Di ethanol | 4.40 | 94.33 | 98.30 |
| | Amine | | | |
| 3. | Acetonitrile | 4.91 | No conversion | — |
| 4. | Ferrous sulfate | 4.79 | No conversion | — |
| | heptahydrate | | | |

### Example 2

In this example, the synthesis of tert-octyl mercaptan was carried out using both a pretreated cation exchange resin catalyst and a fresh cation exchange resin catalyst. A total of 2 grams of catalyst was utilized in a tubular reactor operated in continuous mode. The experiments were conducted at a temperature of 60-110° C. and a pressure of 5-15 bar. Di-isobutylene (DIB) with a purity of 98% and hydrogen sulfide (H₂S) with a purity of 99.9% were employed as the feedstocks. DIB and H₂S were introduced in the reactor after proper mixing, ensuring a mole ratio of H₂S to DIB of 4:1. The experiments were performed over a duration of 24 hours for both the catalyst and the product samples analyzed using gas chromatography. The results of the experiments for both the fresh and pretreated catalysts are presented in Table-4.

TABLE 4
TOM synthesis using feed having 98% DIB

| | | | % TOM | % Heavier | |
|---|---|---|---|---|---|
| Exp | | % DIB | Selec- | oligomers in | % purity |
| No | Catalyst | Conversion | tivity | product | of TOM |
| 1 | Fresh Catalyst | 99.01 | 76.43 | 18.2 | 80.3 |
| 2 | Pretreated | 95.49 | 98.11 | 2.0 | 97.8 |
| | catalyst | | | | |

### Example 3

Similar to Example 1, the activity of both fresh and pretreated cation exchange resin catalysts was evaluated for the formation of tert-dodecyl mercaptan (TDM). The operating conditions were maintained consistent with those described in Example 1. Tri-isobutylene (TIB) with a purity of 90 wt % was used as the feedstock for TDM production. TIB and hydrogen sulfide (H₂S) were fed to reactor maintaining an H₂S to TIB mole ratio of 4:1. The experiments were conducted over a 24-hour period, with product samples analyzed thereafter in the Gas Chromatograph Mass Spectroscopy. The product analysis indicated a significantly lower conversion of TIB compared to DIB, which is attributed to the higher stability of TIB. The results of these experiments are presented in Table 5.

TABLE 5
TDM synthesis using feed having 90% TIB

| | | | % TDM | % Heavier | |
|---|---|---|---|---|---|
| Exp | | % TIB | Selec- | oligomers in | % purity |
| No | Catalyst | Conversion | tivity | product | of TDM |
| 3 | Fresh Catalyst | 10.41 | 93.12 | 31.0 | 92.1 |
| 4 | Pretreated | 7.22 | 94.88 | 7.26 | 94.4 |
| | Catalyst | | | | |

### Example 4

In experiments no. 5-9, the effect of different concentration of MDEA solution on the cation exchange resin active sites and catalyst performance in terms of TOM selectivity has been studied. The pretreatment on cation exchange resin catalyst is done as per the conditions given in Table 2. Experiments were performed with DIB having purity 98% as feed with each pretreated catalyst. The experimental conditions were fixed as per the previous examples. The product samples were analyzed in Gas Chromatograph after each experiment and results are given in Table-6.

TABLE 6
Effect of precursor concentration on Conversion and Selectivity

| | MDEA | | | | |
|---|---|---|---|---|---|
| | concen- | Catalyst Activity | | % TOM | % |
| Exp. | tration | (Acid Capacity), | % DIB | Selec- | Heavier |
| No | (wt %) | meq/g | Conversion | tivity | oligomers |
| 5 | 0 | 5.2 | 99.01 | 76.43 | 18.24 |
| 6 | 2 | 4.4 | 95.50 | 98.30 | 2.00 |
| 7 | 4 | 4.0 | 64.79 | 97.00 | 1.55 |
| 8 | 6 | 3.5 | 41.39 | 97.81 | 1.78 |
| 9 | 8 | 2.9 | No | — | — |
| | | | conversion | | |

### Example 5

Longer duration experiments were performed to assess the performance of the pretreated catalyst. Experiment conditions were kept similar to previous examples. From the outcome of the results, no decrease in catalyst performance observed till 180 hours. The experimental results are shown in FIG. 2.

### ADVANTAGES OF THE PRESENT INVENTION

The present invention provides a process for the production of high purity tert octyl mercaptan & tert dodecyl mercaptan.
The present invention involves the pre-treatment of the cation exchange resin catalyst with appropriate inhibitors such as amine precursors, nitriles and various metals to optimize the catalyst activity so that less undesired by-product formation takes place.
The present invention provides improved yield/selectivity of the desired products tert octyl mercaptan & tert dodecyl mercaptan.

The cation exchange resin catalyst is not deactivated due to less formation of undesired oligomers which generally get deposited on the catalyst surface and reduces the catalyst activity. There is less undesired byproducts formation, thus reducing the load on the product fractionation in purification section.

We claim:

1. A process for producing specialty mercaptans from C₄ oligomerization product, the process comprising:
fractionating C₄ oligomerization product to selectively recover desired oligomer fractions;
loading a cation exchange resin catalyst into a reactor;
pretreating the loaded cation exchange resin catalyst with an inhibitor in the reactor;
feeding the recovered desired oligomer fractions and hydrogen sulfide (H₂S) into the reactor to obtain a reaction product mixture; and
separating the reaction product mixture into an un-converted oligomer and a final product, wherein the final product comprises specialty mercaptans.

2. The process as claimed in claim 1, wherein the cation exchange resin catalyst is pretreated with 2-10 wt % of the inhibitor, followed by heating at a temperature in a range of 50 to 90° C. for 3 to 4 hours, and further followed by drying at a temperature in a range of 100 to 120° C. for 5 to 10 hours in presence of nitrogen (N₂).

3. The process as claimed in claim 1, wherein the recovered desired oligomer fractions and hydrogen sulfide (H₂S) are mixed and reacted in the reactor at a temperature in a range of 50 to 110° C., and at a pressure in a range of 1 to 15 bar.

4. The process as claimed in claim 1, wherein the recovered desired oligomer fractions comprise di-isobutene, and tri-isobutene.

5. The process as claimed in claim 1, wherein the final product comprises tert octyl mercaptan when the oligomer fraction is di-isobutene, and wherein the final product comprises tert dodecyl mercaptan when the oligomer fraction is tri-isobutene.

6. The process as claimed in claim 4, wherein H₂S and di-isobutylene are fed in a mole ratio of 1:1 to 10:1.

7. The process as claimed in claim 4, wherein H₂S and tri-isobutylene are fed in a mole ratio of 1:1 to 10:1.

8. The process as claimed in claim 1, wherein the inhibitor is selected from the group consisting of an amine precursor, a nitrile precursor and a metal precursor.

9. The process as claimed in claim 8, wherein the amine precursor is methyl ethanol amine or methyl di ethanol amine, the nitrile precursor is acetonitrile, and the metal precursor is iron or sodium.

10. The process as claimed in claim 9, wherein methyl di ethanol amine yielded 94.33% conversion of di-isobutylene, and a selectivity of 98.30% tert octyl mercaptan.

11. The process as claimed in claim 1, wherein the cation exchange resin catalyst is an acidic cation exchange resin.

12. The process as claimed in claim 11, wherein the acidic cation exchange resin is sulfonated polystyrene divinyl benzene resin.

13. The process as claimed in claim 1, wherein the pretreated cation exchange resin catalyst has an active site concentration in a range of 3 to 4.5 eq/kg.

14. The process as claimed in claim 5, wherein the di-isobutene conversion to tert octyl mercaptan is in a range of 45% to 98%, and wherein the pretreated cation exchange resin catalyst has a selectivity for tert octyl mercaptan in a range of to 75% to 99%.

15. The process as claimed in claim 5, wherein the tri-isobutene conversion to tert dodecyl mercaptan is in a range of 5% to 10%, and wherein the pretreated cation exchange resin catalyst has a selectivity for tert dodecyl mercaptan in a range of to 75% to 99%.

16. The process as claimed in claim 1, wherein the tert octyl mercaptan has a purity in a range of 75% to 99%.

17. The process as claimed in claim 1, wherein the tert dodecyl mercaptan has a purity in a range of 90% to 95%.
